(19) United States
(12) Patent Application Publication
(10) Pub. No.: US 2010/0174073 A1
(43) Pub. Date: Jul. 8, 2010

(54) PROCESS FOR THE PREPARATION OF ALFUZOSIN AND SALTS THEREOF

(75) Inventors: Uday Rajaram Bapat, Mumbai (IN); Jose Paul Potams, Chennai (IN); Narasimhan Subramanian, Chennai (IN); Jon Valgeirsson, Hafnarfjordur (IS)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: ACTAVIS GROUP PTC EHF, Hafnarfjordur (IS)

(21) Appl. No.: 12/598,839

(22) PCT Filed: May 2, 2008
(86) PCT No.: PCT/IB08/02340

§ 371 Date: Mar. 22, 2010

(30) Foreign Application Priority Data
May 4, 2007 (IN) 955/CHE/2007

(51) Int. Cl. C07D 403/12

(52) U.S. Cl. 544/292

(57) ABSTRACT

The present invention relates to novel N-[3-[(4-acyl-/aroyl-substituted amino-6,7-dimethoxy-2-quinazolinyl)methylamino]propyl]tetrahydro-2-furancarboxamide derivatives, and a process for the preparation thereof. The novel compounds are useful for preparing alfuzosin or a pharmaceutically acceptable salt thereof in high yield and purity.

### CROSS REFERENCE TO RELATED APPLICATION

This application claims priority from Indian Provisional Application Ser. No. 955/CHE/2007 filed May 4, 2007, which is hereby incorporated by reference in its entirety.

### FIELD OF THE DISCLOSURE

The present invention relates to novel N-[3-[(4-acyl-/aroyl-substituted amino-6,7-dimethoxy-2-quinazolinyl)methylamino]propyl]tetrahydro-2-furancarboxamide compounds, and a process for the preparation thereof. The novel compounds are useful for preparing alfuzosin or a pharmaceutically acceptable salt thereof in high yield and purity.

### BACKGROUND OF THE INVENTION

U.S. Pat. No. 4,315,007 discloses 4-amino-6,7-dimethoxyquinazol-2-yl alkylenediamine derivatives and their salts, processes for their preparation, pharmaceutical compositions comprising the derivatives, and method of use thereof. Of these compounds, N-[3-[(4-amino-6,7-dimethoxy-2-quinazolinyl)methylamino]propyl]tetrahydro-2-furancarboxamide is important, since it is well-known as a pharmaceutically active substance under the name of Alfuzosin. Alfuzosin is a selective antagonist of post-synaptic α-adrenoreceptors and is indicated for the treatment of the signs and symptoms of benign prostatic hyperplasia. It is available in the market from Sanofi-Aventis under the brand name ‘Uroxatral’ as extended release tablets containing 10 mg alfuzosin hydrochloride as the active ingredient. Alfuzosin is represented by the following structural formula I:

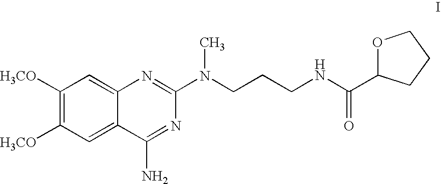

Processes for the preparation of alfuzosin and its pharmaceutically acceptable salts are disclosed in U.S. Pat. No. 4,315,007 and GB Patent No. 2231571.

According to U.S. Pat. No. 4,315,007 (herein after referred to as the '007 patent), alfuzosin hydrochloride is prepared by the reaction of 4-amino-2-chloro-6,7-dimethoxyquinazoline with 3-methylaminopropionitrile in isoamyl alcohol to produce N-(4-amino-6,7-dimethoxyquinazol-2-yl)-N-methyl-2-cyanoethylamine, which is then hydrogenated in the presence of Raney nickel followed by treatment with hydrochloric acid to produce N₁-(4-amino-6,7-dimethoxyquinazol-2-yl)-N₁-methylpropylenediamine hydrochloride intermediate. This intermediate is further reacted with tetrahydrofuroic acid to produce alfuzosin hydrochloride.

Alfuzosin or a pharmaceutically acceptable salts thereof obtained by the process described in the '007 patent does not have satisfactory purity.

U.S. Pat. No. 5,545,738 (herein after referred to as the '738 patent) discloses a process for preparing alfuzosin hydrochloride dihydrate. In general, the process of the '738 patent includes crystallizing anhydrous alfuzosin hydrochloride in a mixture of acetone:water (4:1) to provide alfuzosin hydrochloride dihydrate. The '738 patent further discloses anhydrous, dihydrate, triydrate and tetrahydrate crystalline forms of alfuzosin hydrochloride.

PCT Publication No. WO 2006/030449 A1 (herein after referred to as the '449 application) discloses a process for preparing crystalline alfuzosin base and use thereof for the preparation of alfuzosin hydrochloride. The process used in the '449 application suffers from disadvantages such as the use of additional solvents, multiple crystallizations steps to isolate alfuzosin base and thus resulting low overall yields of the product.

Like any synthetic compound, alfuzosin salts can contain extraneous compounds or impurities that can come from many sources. They can be unreacted starting materials, by-products of the reaction, products of side reactions, or degradation products. Impurities in alfuzosin or any active pharmaceutical ingredient (API) are undesirable and, in extreme cases, might even be harmful to a patient being treated with a dosage form containing the API.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (“ICH”) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the API, alfuzosin, it must be analyzed for purity, typically, by HPLC, TLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use.

A need remains for an improved and commercially viable process of preparing pharmaceutically acceptable salts of alfuzosin with high purity and high yield without isolating alfuzosin free base as solid to resolve the problems associated with the processes described in the prior art, and that will be suitable for large-scale preparation. Desirable process properties include less hazardous and environmentally friendly reagents, reduced cost, and greater simplicity and increased purity of the product.

One object of the present invention is to provide an improved process for the preparation of highly pure alfuzosin or a pharmaceutically acceptable salt thereof.

Another object of the invention is to provide a novel N-[3-[(4-acyl-/aroyl-substituted-amino-6,7-dimethoxy-2-quinazolinyl)methylamino]propyl]tetrahydro-2-furancarboxamide compounds and a process for the preparation thereof.

Yet another object of the present invention is to provide a process for preparing highly pure alfuzosin or a pharmaceutically acceptable salt thereof by using the N-[3-[(4-acyl-/aroyl-substituted-amino-6,7-dimethoxy-2-quinazolinyl)methylamino]propyl]tetrahydro-2-furancarboxamide compounds, which is simple and easy to handle and cost effective.

### SUMMARY OF THE INVENTION

In one aspect, the present invention encompasses novel compounds N-[3-[(4-acyl-/aroyl-substituted amino-6,7-dimethoxy-2-quinazolinyl)methylamino]propyl]tetrahydro-2-furancarboxamide, denominated diamide compound II, having the following structural formula:

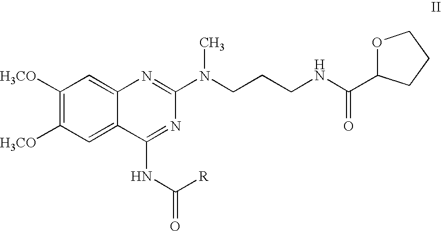

wherein R is C₁₋₁₂ straight or branched chain alkyl, cycloalkyl, haloalkyl, or substituted or unsubstituted aryl.

Preferably R is methyl, ethyl, propyl, isopropyl, isobutyl, tert-butyl, chloromethyl, phenyl, toulyl, benzyl, p-nitrobenzyl, dibromophenyl, toluene sulfonyl or p-methoxybenzyl.

In another aspect, the present invention is directed to a process for preparing the diamide compound of formula II by reaction of alfuzosin with a suitable activating agent selected from the group comprising acid anhydrides, mixed anhydrides and acid chlorides, and in a suitable solvent.

In another aspect, the present invention encompasses a process for preparing the highly pure alfuzosin or a pharmaceutically acceptable salt thereof using the novel diamide compounds.

In a preferred aspect, the present invention encompasses an efficient, simple and commercially viable process for preparing highly pure alfuzosin hydrochloride without isolating alfuzosin base as a solid.

In another aspect, the present invention provides alfuzosin or a pharmaceutically acceptable salt thereof having total purity of greater than about 99.7%, specifically greater than about 99.9%, and more specifically greater than about 99.95% measured by HPLC.

In another aspect, a preferred compound of formula II prepared by the process described herein is N-[3-[(4-acetylamino-6,7-dimethoxy-2-quinazolinyl)methylamino]propyl]tetrahydro-2-furancarboxamide, denominated N-acetyl alfuzosin, of formula II(a) (formula II, wherein R is methyl):

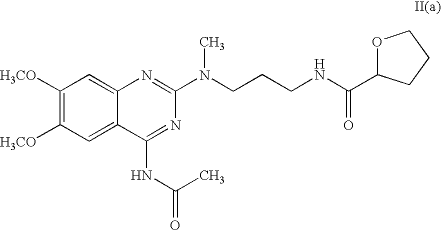

In another aspect, another preferred compound of formula II prepared by the process described herein is N-[3-[(4-propionylamino-6,7-dimethoxy-2-quinazolinyl)methylamino]propyl]tetrahydro-2-furancarboxamide, denominated N-propionyl alfuzosin, of formula II(b) (formula II, wherein R is ethyl):

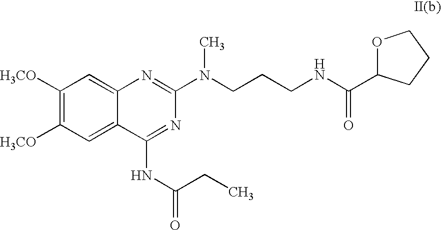

Advantageously, the reagents used for present invention are less hazardous and easy to handle at commercial scale and also involves less expensive reagents.

### DETAILED DESCRIPTION OF THE INVENTION

According to one aspect of the present invention, there is provided novel compounds N-[3-[(4-acyl-/aroyl-substituted amino-6,7-dimethoxy-2-quinazolinyl)methyl amino]propyl]tetrahydro-2-furancarboxamide, denominated diamide compound II, having the following structural formula:

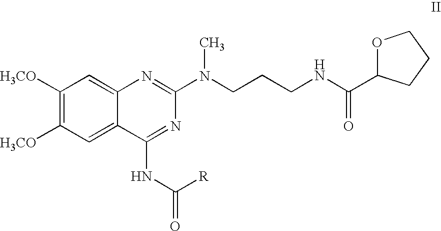

wherein R is C₁₋₁₂ straight or branched chain alkyl, cycloalkyl, haloalkyl, or substituted or unsubstituted aryl.

Preferably R is methyl, ethyl, propyl, isopropyl, isobutyl, tert-butyl, chloromethyl, phenyl, toulyl, benzyl, p-nitrobenzyl, dibromophenyl, toluene sulfonyl, or p-methoxybenzyl.

In another aspect, a preferred compound of formula II prepared by the process described herein is N-[3-[(4-acetylamino-6,7-dimethoxy-2-quinazolinyl)methylamino]propyl]tetrahydro-2-furancarboxamide, denominated N-acetyl alfuzosin, of formula II(a) (formula II, wherein R is methyl):

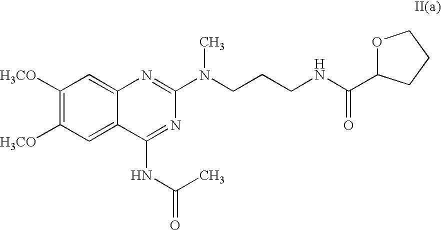

In another aspect, another preferred compound of formula II prepared by the process described herein is N-[3-[(4-propionylamino-6,7-dimethoxy-2-quinazolinyl)methylamino]propyl]tetrahydro-2-furancarboxamide, denominated N-propionyl alfuzosin, of formula II(b) (formula II, wherein R is ethyl):

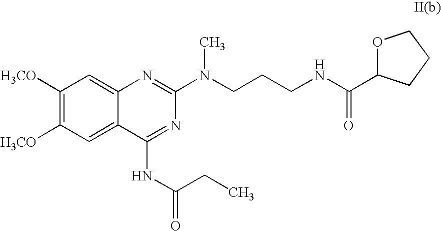

It has been surprisingly found that the novel diamide compounds are useful intermediates for preparing alfuzosin and its pharmaceutically acceptable salts in high purity.

According to another aspect of the present invention, there is provided a process for the preparation of diamide Compound of formula II:

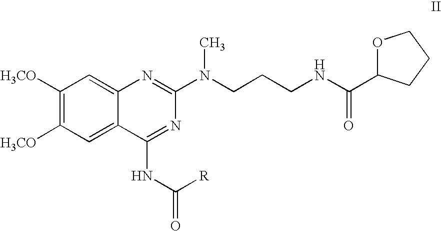

wherein R is C₁₋₁₂ straight or branched chain alkyl, cycloalkyl, haloalkyl, or substituted or unsubstituted aryl;
which comprises:
reacting alfuzosin of formula I:

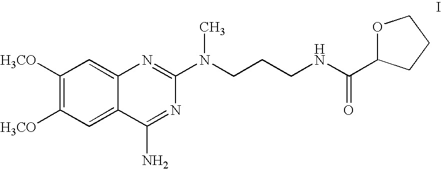

with a suitable activating agent selected from the group comprising an acid anhydride of formula III(a), a mixed anhydride of formula III(b) and an acid chloride of formula III(c):

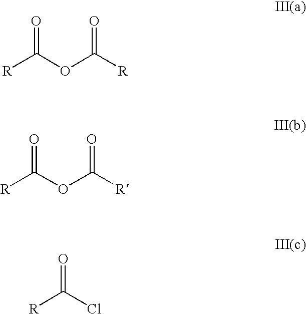

wherein R is as defined in formula II; and R′ is alkoxy or imidazolyl; in a suitable solvent to produce the diamide compound of formula II.

Preferably R is methyl, ethyl, propyl, isopropyl, isobutyl, tert-butyl, chloromethyl, phenyl, toulyl, benzyl, p-nitrobenzyl, dibromophenyl, toluene sulfonyl, or p-methoxybenzyl, and more preferably R is methyl, ethyl or propyl.

The suitable solvents include, but are not limited to, hydrocarbons, chlorinated hydrocarbons, ketones, polar aprotic solvents, ethers, nitriles, esters, and the like, and mixtures thereof. Exemplary hydrocarbon solvents include, but are not limited to, pentane, hexane, heptane and isomers thereof, cyclohexane, toluene, xylene, and mixtures thereof. Specific hydrocarbon solvents are toluene and cyclohexane. Exemplary chlorinated hydrocarbon solvents include, but are not limited to, methylene chloride, ethyl dichloride, chloroform and carbon tetrachloride or mixtures thereof. Specific chlorinated hydrocarbon solvent is methylene chloride. Exemplary ketone solvents include, but are not limited to, acetone, methyl isobutyl ketone, and the like, and mixtures thereof. Exemplary polar aprotic solvents include, but are not limited to, N,N-dimethylformamide, N,N-dimethylacetamide, dimethylsulfoxide, and mixtures thereof. Exemplary ether solvents include, but are not limited to, diisopropyl ether, methyl tert-butyl ether, tetrahydrofuran, dioxane, and the like, and mixtures thereof. Exemplary nitrile solvents include, but are not limited to, acetonitrile, and the like, and mixtures thereof. Exemplary ester solvents include, but are not limited to, ethyl acetate, isopropyl acetate, and the like and mixtures thereof.

Preferable solvents are hydrocarbons, chlorinated hydrocarbons, and mixtures thereof, and more preferably hexane, heptane, cyclohexane, toluene, methylene chloride, and mixtures thereof.

The reaction is preferably carried out at a temperature of 0° C. to the reflux temperature of the solvent used for at least 1 hour, more preferably at a temperature of about 25° C. to the reflux temperature of the solvent used from about 1 hour to about 30 hours, and most preferably at the reflux temperature of the solvent used from about 10 hours to about 25 hours.

In an embodiment, the activating agent in the molar ratio of about 1 to 10 moles, specifically about 2 to 5 moles, per 1 mole of alfuzosin of formula I is used in order to ensure a proper course of the reaction.

In one embodiment, the diamide compound of formula II obtained is isolated as solid from a suitable organic solvent by methods usually known, in the art such as cooling, partial removal of the solvent from the solution, addition of precipitating solvent, or a combination thereof. Suitable solvents include, but are not limited to, alcohols, hydrocarbons, ketones, cyclic ethers, aliphatic ethers, nitriles, alkane solvents, and the like, and mixtures thereof. Preferable solvents are hexane, heptane, cyclohexane, toluene, methylene chloride, acetone, and mixtures thereof.

According to another aspect of the present invention, there is provided a process for the preparation of highly pure N-[3-[(4-amino-6,7-dimethoxy-2-quinazolinyl)methyl amino]propyl]tetrahydro-2-furancarboxamide (Alfuzosin) or a pharmaceutically acceptable salt thereof, which comprises:
a) reacting N-(4-amino-6,7-dimethoxyquinazol-2-yl)-N-methylpropylenediamine with tetrahydro-2-furoic acid in the presence of N,N-carbonyldiimidazole in a suitable organic solvent to obtain a solution containing alfuzosin free base of formula I:

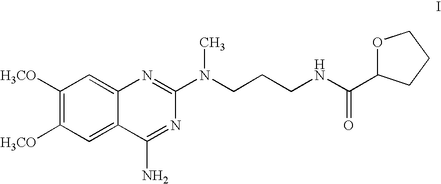

b) optionally, filtering the solution obtained in step-(a) to remove any extraneous matter;
c) optionally, partially or completely concentrating the solution obtained in step-(a) or step-(b) to produce a solution containing alfuzosin free base and the suitable organic solvent;
d) reacting the alfuzosin solution obtained in step-(a), step-(b) or step-(c) with a suitable activating agent selected from the group comprising an acid anhydride of formula III(a), a mixed anhydride of formula III(b) and an acid chloride of formula III(c):

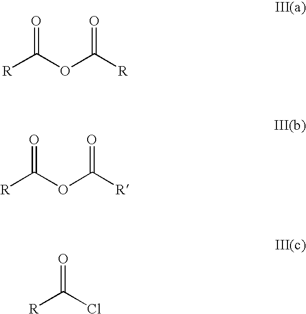

wherein R is C₁₋₁₂ straight or branched chain alkyl, cycloalkyl, haloalkyl, or substituted or unsubstituted aryl; and R′ is alkoxy or imidazolyl;
to produce a diamide compound of formula II:

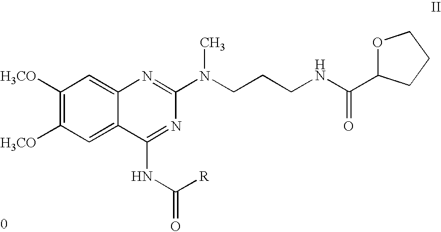

wherein R is as defined above;
e) hydrolyzing the compound of formula II to produce a reaction mass containing alfuzosin or a salt thereof; and
f) isolating pure alfuzosin or a pharmaceutically acceptable salt thereof.

Preferably ‘R’ in the compounds of formulae II, III(a), III(b) & III(c) is methyl, ethyl, propyl, isopropyl, isobutyl, tert-butyl, chloromethyl, phenyl, toulyl, benzyl, p-nitrobenzyl, dibromophenyl, toluene sulfonyl, or p-methoxybenzyl, and more preferably R is methyl, ethyl or propyl.

The suitable organic solvent used in step-(a) is selected from the group comprising hydrocarbons, chlorinated hydrocarbons, and mixtures thereof. Preferable organic solvents are selected from the group consisting of hexane, heptane, cyclohexane, toluene, methylene chloride, and mixtures thereof, and more preferably methylene chloride.

The reaction in step-(d) can be carried out in the presence or absence of any additional solvents. The suitable solvents include, but are not limited to, hydrocarbons, chlorinated hydrocarbons, ketones, polar aprotic solvents, ethers, nitriles, esters, and the like, and mixtures thereof. Exemplary hydrocarbon solvents include, but are not limited to, pentane, hexane, heptane and isomers thereof, cyclohexane, toluene, xylene, and mixtures thereof. Specific hydrocarbon solvents are toluene and cyclohexane. Exemplary chlorinated hydrocarbon solvents include, but are not limited to, methylene chloride, ethyl dichloride, chloroform and carbon tetrachloride or mixtures thereof. Specific chlorinated hydrocarbon solvent is methylene chloride. Exemplary ketone solvents include, but are not limited to, acetone, methyl isobutyl ketone, and the like, and mixtures thereof. Exemplary polar aprotic solvents include, but are not limited to, N,N-dimethylformamide, N,N-dimethylacetamide, dimethylsulfoxide, and mixtures thereof. Exemplary ether solvents include, but are not limited to, diisopropyl ether, methyl tert-butyl ether, tetrahydrofuran, dioxane, and the like, and mixtures thereof. Exemplary nitrile solvents include, but are not limited to, acetonitrile, and the like, and mixtures thereof. Exemplary ester solvents include, but are not limited to, ethyl acetate, isopropyl acetate, and the like and mixtures thereof.

Preferable solvents are hydrocarbons, chlorinated hydrocarbons, and mixtures thereof, and more preferably hexane, heptane, cyclohexane, toluene, methylene chloride, and mixtures thereof.

The reaction in step-(d) is preferably carried out at a temperature of 0° C. to the reflux temperature of the solvent used for at least 1 hour, more preferably at a temperature of about 25° C. to the reflux temperature of the solvent used from about 1 hour to about 30 hours, and most preferably at the reflux temperature of the solvent used from about 10 hours to about 25 hours.

In an embodiment, the activating agent in the molar ratio of about 1 to 10 moles, specifically about 2 to 5 moles, per 1 mole of alfuzosin of formula I is used in order to ensure a proper course of the reaction.

The diamide compound of formula II obtained in step-(d) may by used directly in the next step or the compound of formula II may by isolated from the reaction medium and then used in the next step.

In a preferred embodiment, the diamide compound of formula II obtained in step-(d) is isolated as solid from a suitable organic solvent by methods usually known in the art such as cooling, partial removal of the solvent from the solution, addition of precipitating solvent, or a combination thereof. Suitable solvents include, but are not limited to, alcohols, hydrocarbons, ketones, cyclic ethers, aliphatic ethers, nitriles, alkanes, and the like, and mixtures thereof. Preferable solvents are hexane, heptane, cyclohexane, toluene, methylene chloride, acetone, and mixtures thereof.

The hydrolysis in step-(e) can be performed by using an acid or a base. The base can be an organic or inorganic. Exemplary inorganic bases include, but are not limited to, hydroxides, carbonates and bicarbonates of alkali or alkaline earth metals. Specific inorganic bases are sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide, and more specifically sodium hydroxide, potassium hydroxide, sodium carbonate and potassium carbonate. The base hydrolysis is usually carried out at 0° C. to reflux temperature in presence or absence of solvents.

The hydrolysis is preferably carried out by using an acid. The acid can be an organic or inorganic acid. Preferable inorganic acids include, but are not limited to, hydrochloric acid, hydrobromic acid, sulfuric acid, phosphoric acid, and the like. Preferable organic acids include, but are not limited to, p-toluenesulfonic, methanesulfonic acid, oxalic acid, p-bromophenylsulfonic acid, carbonic acid, succinic acid, citric acid, benzoic acid, acetic acid, fumaric acid and the like. The acid hydrolysis is usually carried out at a temperature of about 25° C. to the reflux temperature of the solvent used.

Exemplary solvents for step-(e) include, but are not limited to, water, alcohols, ketones, cyclic ethers, aliphatic ethers, hydrocarbons, chlorinated hydrocarbons, nitriles, esters and the like, and mixtures thereof. Preferable solvents are water, alcohols, ketones, and mixtures thereof, and more preferably water, methanol, ethanol, isopropyl alcohol, tert-butanol, acetone and mixtures thereof.

If the hydrolysis reaction is carried out in the presence of a base the product obtained is alfuzosin base, which is in-situ, converted into a pharmaceutically acceptable acid addition salt of alfuzosin using a suitable acid in a suitable solvent. Preferably, the pharmaceutically acceptable acid addition salts of alfuzosin can be obtained directly in step-(e) by carrying out the hydrolysis reaction in the presence of a suitable acid.

The suitable acids include, but are not limited to, hydrochloric acid, hydrobromic acid, hydroiodic acid, acetic acid, fumaric acid, tartaric acid, succinic acid, methanesulfonic acid, and more preferable acid is hydrochloric acid.

Hydrochloric acid used may be in the form of aqueous hydrochloric acid or in the form of hydrogen chloride gas or hydrogen chloride dissolved in an organic solvent. The organic solvent used for dissolving hydrogen chloride gas or hydrogen chloride is selected from the group consisting of ethanol, methanol, isopropyl alcohol, ethyl acetate, diethyl ether, dimethyl ether and acetone.

The isolation in step-(f) is initiated by methods usually known in the art such as cooling, seeding, partial removal of the solvent from the solution, by adding an anti-solvent to the solution, or a combination thereof.

Preferably the isolation is carried out by cooling the solution at a temperature of below 30° C., and more preferably at about 0° C. to about 25° C. The solid obtained is collected by filtration or centrifugation.

In a preferred aspect of the present invention, there is provided an efficient, simple and commercially viable process for preparing highly pure alfuzosin hydrochloride without isolating alfuzosin base as a solid, comprising:
a) reacting N-(4-amino-6,7-dimethoxyquinazol-2-yl)-N-methylpropylenediamine with tetrahydro-2-furoic acid in the presence of N,N-carbonyldiimidazole in a suitable organic solvent to obtain a solution containing alfuzosin free base;
b) optionally, filtering the solution obtained in step-(a) to remove any extraneous matter;
c) optionally, partially or completely concentrating the solution obtained in step-(a) or step-(b) to produce a solution containing alfuzosin free base and the suitable organic solvent;
d) reacting the alfuzosin solution obtained in step-(a), step-(b) or step-(c) with an acylating agent selected from acetic anhydride or propionic anhydride to produce appropriate N-acyl alfuzosin;
e) hydrolyzing the N-acyl alfuzosin obtained in step-(d) with methanolic hydrochloride in a suitable solvent to produce a reaction mass containing alfuzosin hydrochloride; and
f) isolating pure alfuzosin hydrochloride.

The suitable organic solvent used in step-(a) is selected from the group comprising hydrocarbons, chlorinated hydrocarbons, and mixtures thereof. Preferable organic solvents used in step-(a) are selected from the group consisting of hexane, heptane, cyclohexane, toluene, methylene chloride, and mixtures thereof, and more preferably methylene chloride.

The reaction in step-(d) can be carried out in the presence or absence of any additional solvents. Preferable solvents include, but are not limited to, hydrocarbons, chlorinated hydrocarbons, and mixtures thereof, and more preferably hexane, heptane, cyclohexane, toluene, methylene chloride, and mixtures thereof.

The reaction in step-(d) is preferably carried out at a temperature of 0° C. to the reflux temperature of the solvent used for at least 1 hour, more preferably at a temperature of about 25° C. to the reflux temperature of the solvent used from about 1 hour to about 30 hours, and most preferably at the reflux temperature of the solvent used from about 10 hours to about 25 hours.

In an embodiment, the acylating agent in the molar ratio of about 1 to 6 moles, specifically about 3 to 5 moles, per 1 mole of alfuzosin is used in order to ensure a proper course of the reaction.

The N-acyl alfuzosin compound obtained in step-(d) may by used directly in the next step or the compound of formula II may by isolated from the reaction medium and then used in the next step.

In a preferred embodiment, the N-acyl alfuzosin compound obtained in step-(d) is isolated as solid from a suitable organic solvent by methods usually known in the art such as cooling, partial removal of the solvent from the solution, addition of precipitating solvent, or a combination thereof. Suitable solvents include, but are not limited to, alcohols, hydrocarbons, ketones, cyclic ethers, aliphatic ethers, nitriles, alkanes, and the like, and mixtures thereof. Preferable solvents are hexane, heptane, cyclohexane, toluene, methylene chloride, acetone, and mixtures thereof.

The hydrolysis is usually carried out at a temperature of about 25° C. to the reflux temperature of the solvent used.

Exemplary solvents for step-(e) include, but are not limited to, water, alcohols, ketones, cyclic ethers, aliphatic ethers, hydrocarbons, chlorinated hydrocarbons, nitriles, esters and the like, and mixtures thereof. Preferable solvents are water, alcohols, ketones, and mixtures thereof, and more preferably water, methanol, ethanol, isopropyl alcohol, tert-butanol, acetone and mixtures thereof.

The isolation in step-(f) is initiated by methods usually known in the art such as cooling, seeding, partial removal of the solvent from the solution, by adding an anti-solvent to the solution, or a combination thereof.

Preferably the isolation is carried out by cooling the solution at a temperature of below 30° C., and more preferably at about 0° C. to about 25° C. The solid obtained is collected by filtration or centrifugation.

The alfuzosin or a pharmaceutically acceptable salt thus obtained may be further or additionally dried to achieve the desired residual solvent values. For example, the product may be further or additionally dried in a tray drier, or dried under vacuum and/or in a Fluid Bed Drier. If desired, the solution containing the product can be treated with activated charcoal and filtered while hot or the slurry containing the pure product may be cooled prior to filtration.

Alfuzosin or a pharmaceutically acceptable salt obtained by the process disclosed herein, have a total purity of greater than about 99.7%, specifically greater than about 99.9%, and more specifically greater than about 99.95% measured by HPLC.

The term “crude” in the specification refers to alfuzosin or a pharmaceutically acceptable salt thereof having HPLC purity less than 96%.

### EXPERIMENTAL

The purity of Alfuzosin Hydrochloride was measured by HPLC under the following conditions:
Column: (Inertsil ODS2, 150×4.6 mm×5 μm)
Column temperature: 30° C.
Mobile phase: Buffer: Acetonitrile:THF (80:20:1.0)
Preparation of Buffer: (Sodium perchlorate solution): Take 5.0 ml of perchloric acid in 1000 ml beaker contains 900 ml of HPLC water. Adjust the pH of this solution to 3.5 with diluted NaOH solution and transfer this total solution to 1000 ml measuring cylinder and make up to the mark with HPLC water. Filter this solution using 0.45 μm membrane filter.
Flow rate: (1.0 ml/min; Injection volume: 20 μl)
Run time: (60 minutes)
Retention time: (Alfuzosin hydrochloride about 7.0 minutes)

The following examples are given for the purpose of illustrating the present disclosure and should not be considered as limitation on the scope or spirit of the disclosure.

### EXAMPLES

### Example 1

### Preparation of N-[3-[(4-acetylamino-6,7-dimethoxy-2-quinazolinyl)methylamino]propyl]tetrahydro-2-furancarboxamide (N-Acetyl Alfuzosin)

A mixture of N-(4-amino-6,7-dimethoxyquinazol-2-yl)-N-methyl-2-cyanoethylamine hydrochloride (55 gm), saturated methanolic ammonia (550 ml) and Raney nickel (82.5 gm) was taken into a pressure vessel, and hydrogenated under 10 kg pressure. The reaction mass was heated to 80° C. and maintained for 10 hours. The resulting mass was cooled to 40° C., filtered the catalyst and washed with methanol (506 ml). The filtrate was distilled to give N-(4-amino-6,7-dimethoxyquinazol-2-yl)-N-methylpropylenediamine. The diamine compound is reacted with tetrahydro-2-furoic acid (18.2 ml) in presence of N,N-carbonyldiimidazole (30.8 gm) in dichloromethane (755 ml) at 40° C. for 4 hours to produce a reaction mass containing N-[3-[(4-amino-6,7-dimethoxy-2-quinazolinyl)methylamino]propyl]tetrahydro-2-furancarboxamide. The reaction mass was cooled to 35° C., washed with water (1000 ml) and the dichloromethane layer dried with anhydrous sodiumsulfate (70 gm). The resulting dichloromethane layer was distilled to half of the initial volume, followed by the addition of acetic anhydride (38 ml) and the resulting mixture was stirred at reflux temperature (45° C.) for 24 hours. The dichloromethane was distilled off under vacuum. This was followed by the addition of acetone (600 ml) and the resulting slurry was filtered and washed with acetone (200 ml) to produce the title compound (Yield: 80%; HPLC Purity=98%)

### Example 2

### Preparation of Alfuzosin Hydrochloride

N-Acetyl alfuzosin (40.0 gm) was dissolved in methanol (120 ml). The resulting solution was acidified with methanolic hydrochloride (27.68 ml). The reaction mixture was heated at 40° C. for 8 hours. The resulting mass was cooled at 25° C. The separated solid was filtered under nitrogen atmosphere, washed with methanol (75 ml) and then dried at 80-85° C. in vacuum to produce the title compound (Yield: 90%; HPLC Purity: 99.90%).

### Example 3

### Preparation of Alfuzosin Hydrochloride

N-Acetyl alfuzosin (40.0 gm) was dissolved in isoamyl alcohol (207.66 ml). The resulting solution was acidified with methanolic hydrochloride (52.8 ml). The reaction mixture was heated at 40° C. for 16 hours. The resulting mass was cooled at 25° C. The separated solid was filtered under nitrogen atmosphere and washed with isoamyl alcohol (197.7 ml). The resulting wet cake was refluxed at 78° C. with ethyl acetate (280.48 ml) for 30 minutes. The resulting solid was filtered, washed with ethyl acetate (117.51 ml) and then dried under vacuum at 110° C. to produce the title compound (Yield: 90%; HPLC Purity: 99.93%; and Content of N-Acetyl alfuzosin impurity: 0.04%). The anhydrous alfuzosin hydrochloride isolated is confirmed to polymorph Form I.

### Example 4

### Preparation of N-[3-[(4-propionylamino-6,7-dimethoxy-2-quinazolinyl)methyl amino]propyl]tetrahydro-2-furancarboxamide (N-Propionyl Alfuzosin)

Propionic anhydride (17.47 ml) was added to a solution of alfuzosin base (11.7 gm) in dichloromethane (35 ml). The reaction mixture was stirred at 40° C. for 24 hours. The dichloromethane was distilled off under vacuum. This was followed by the addition of hexane (100 ml) and the resulting slurry was filtered and washed with hexane (50 ml) to give the title compound (Yield: 89%; HPLC Purity: 98%).

### Example 5

### Preparation of Alfuzosin Hydrochloride

N-Propionyl alfuzosin (5.0 gm) was dissolved in n-butanol (25 ml). The resulting solution was acidified with methanolic hydrochloride (9.5 ml). The reaction mixture was heated at 40° C. for 6 hours. The resulting mass was cooled at 25° C. The separated solid was filtered under nitrogen atmosphere, washed with n-butanol (25 ml) and then dried at 110° C. under vacuum to give title compound (Yield: 80%; HPLC Purity: 99.8%).

1. A diamide compound, N-[3-[(4-acyl-/aroyl-substituted amino-6,7-dimethoxy-2-quinazolinyl)methylamino]propyl]tetrahydro-2-furancarboxamide, having the structural formula II: 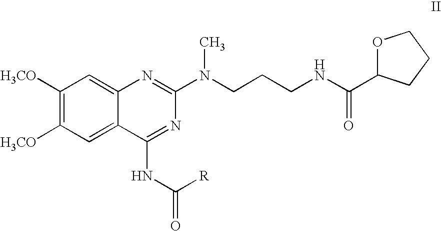
wherein R is C₁₋₁₂ straight or branched chain alkyl, cycloalkyl, haloalkyl, or substituted or unsubstituted aryl.

2. The compound of claim 1, wherein the R is methyl, ethyl, propyl, isopropyl, isobutyl, tert-butyl, chloromethyl, phenyl, toulyl, benzyl, p-nitrobenzyl, dibromophenyl, toluene sulfonyl or p-methoxybenzyl.

3. A process for preparing the compound of claim 1, comprising: reacting alfuzosin of formula I: 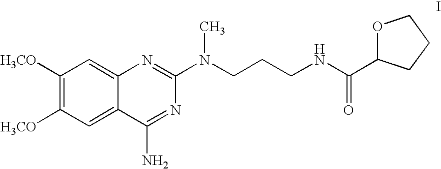
with a suitable activating agent selected from the group comprising an acid anhydride of formula III(a), a mixed anhydride of formula III(b) and an acid chloride of formula III(c): 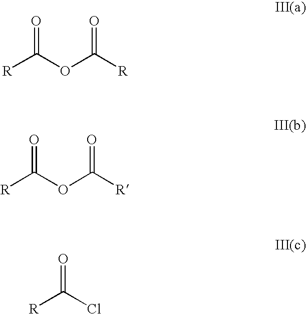
wherein R is C₁₋₁₂ straight or branched chain alkyl, cycloalkyl, haloalkyl, or substituted or unsubstituted aryl; and R′ is alkoxy or imidazolyl; in a suitable solvent to produce the diamide compound of formula II.

4. The process of claim 3, wherein R is methyl, ethyl, propyl, isopropyl, isobutyl, tert-butyl, chloromethyl, phenyl, toulyl, benzyl, p-nitrobenzyl, dibromophenyl, toluene sulfonyl or p-methoxybenzyl.

5. The process of claim 4, wherein R is methyl, ethyl or propyl.

6. The process of claim 3, wherein the solvent is selected from the group consisting of hydrocarbons, chlorinated hydrocarbons, ketones, polar aprotic solvents, ethers, nitriles, esters, and mixtures thereof.

7. The process of claim 6, wherein the solvent is selected from the group consisting of hexane, heptane, cyclohexane, toluene, methylene chloride, and mixtures thereof.

8. The process of claim 3, wherein the reaction is carried out at a temperature of 0° C. to the reflux temperature of the solvent used.

9. The process of claim 3, wherein the activating agent is used in the molar ratio of about 1 to 10 moles per 1 mole of alfuzosin of formula I.

10. The process of claim 9, wherein the activating agent is used in the molar ratio of about 2 to 5 moles per 1 mole of alfuzosin of formula I.

11. The process of claim 3, wherein the diamide compound obtained is isolated as solid from a suitable organic solvent by cooling, partial removal of the solvent from the solution, addition of precipitating solvent, or a combination thereof, and wherein the organic solvent is selected from the group consisting of alcohols, hydrocarbons, ketones, cyclic ethers, aliphatic ethers, nitriles, alkanes, and mixtures thereof.

12. (canceled)

13. The process of claim 11, wherein the organic solvent is selected from the group consisting of hexane, heptane, cyclohexane, toluene, methylene chloride, acetone, and mixtures thereof.

14. A process for preparing highly pure alfuzosin or a pharmaceutically acceptable salt thereof comprising:
a) reacting N-(4-amino-6,7-dimethoxyquinazol-2-yl)-N-methylpropylenediamine with tetrahydro-2-furoic acid in the presence of N,N-carbonyldiimidazole in a suitable organic solvent to obtain a solution containing alfuzosin free base of formula I: 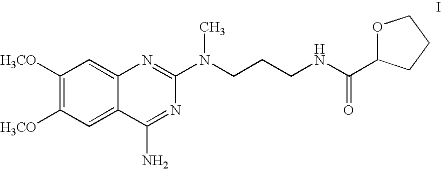
b) optionally, filtering the solution obtained in step-(a) to remove any extraneous matter;
c) optionally, partially or completely concentrating the solution obtained in step-(a) or step-(b) to produce a solution containing alfuzosin free base and the suitable organic solvent;
d) reacting the alfuzosin solution obtained in step-(a), step-(b) or step-(c) with a suitable activating agent selected from the group comprising an acid anhydride of formula III(a), a mixed anhydride of formula III(b) and an acid chloride of formula III(c): 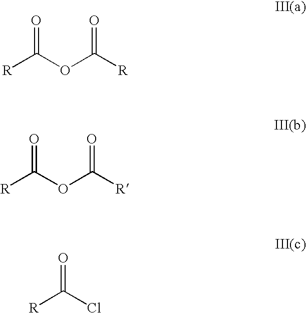
wherein R is C₁₋₁₂ straight or branched chain alkyl, cycloalkyl, haloalkyl, or substituted or unsubstituted aryl; and R′ is alkoxy or imidazolyl;
to produce a diamide compound of formula II: 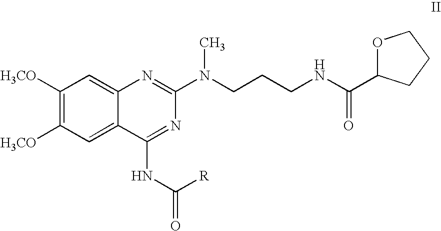
wherein R is as defined above;
e) hydrolyzing the compound of formula II to produce a reaction mass containing alfuzosin or a salt thereof; and
f) isolating pure alfuzosin or a pharmaceutically acceptable salt thereof.

15. The process of claim 14, wherein R in the compounds of formulae II, III(a), III(b) & III(c) is methyl, ethyl, propyl, isopropyl, isobutyl, tert-butyl, chloromethyl, phenyl, toulyl, benzyl, p-nitrobenzyl, dibromophenyl, toluene sulfonyl, or p-methoxybenzyl.

16. The process of claim 15, wherein the R is methyl, ethyl or propyl.

17. The process of claim 14, wherein the organic solvent used in step-(a) is selected from the group consisting of hydrocarbons, chlorinated hydrocarbons, and mixtures thereof, wherein the reaction in step-(d) is carried out in the presence a solvent selected from the group consisting of hydrocarbons, chlorinated hydrocarbons, ketones, polar aprotic solvents, ethers, nitriles, esters, and mixtures thereof, and wherein the solvent used in step-(e) is selected from the group consisting of water, alcohols, ketones, cyclic ethers, aliphatic ethers, hydrocarbons, chlorinated hydrocarbons, nitriles, esters, and mixtures thereof.

18. The process of claim 17, wherein the organic solvent used in step-(a) is methylene chloride, wherein the solvent used in step-(d) is selected from the group consisting of hexane cyclohexane, toluene, methylene chloride, and mixtures thereof, and wherein the solvent used in step-(e) is selected from the group consisting of water, methanol, ethanol, isopropyl alcohol, tort-butanol, acetone, and mixtures thereof.

19. (canceled)

20. (canceled)

21. The process of claim 14, wherein the hydrolysis in step-(e) is performed by using an acid or a base, wherein the acid is an organic or inorganic acid, and wherein the base is an organic or inorganic base.

22. (canceled)

23. (canceled)

24. The process of claim 21, wherein the acid is selected form the group consisting of hydrochloric acid, hydrobromic acid, sulfuric acid, phosphoric acid p-toluenesulfonic acid, methanesulfonic acid, oxalic acid, p-bromophenylsulfonic acid, carbonic acid, succinic acid, citric acid, benzoic acid, acetic acid, fumaric acid, and wherein the base is selected from the group consisting of hydroxides, carbonates and bicarbonates of alkali or alkaline earth metals.

25. (canceled)

26. (canceled)

27. (canceled)

28. (canceled)

29. The process of claim 14, wherein the pharmaceutically acceptable acid addition salts of alfuzosin are obtained directly in step-(e) by carrying out the hydrolysis reaction in the presence of a suitable acid, wherein the suitable acid is selected from the group consisting of hydrochloric acid, hydrobromic acid, hydroiodic acid, acetic acid, fumaric acid, tartaric acid, succinic acid, and methanesulfonic acid.

30. (canceled)

31. The process of claim 29, wherein the acid is hydrochloric acid, wherein the hydrochloric acid is used in the form of an aqueous hydrochloric acid or in the form of hydrogen chloride gas or hydrogen chloride dissolved in an organic solvent, and wherein the organic solvent is selected from the group consisting of ethanol, methanol, isopropyl alcohol, ethyl acetate, diethyl ether, dimethyl ether and acetone.

32. (canceled)

33. (canceled)

34. The process of claim 14, wherein the isolation in step-(f) is initiated by cooling, seeding, partial removal of the solvent from the solution, by adding an anti-solvent to the solution, or a combination thereof.

35. The process of claim 34, wherein the isolation is carried out by cooling the solution at a temperature of below 30° C.

36. N-Acetyl substituted compound of alfuzosin, N-[3-[(4-acetylamino-6,7-dimethoxy-2-quinazolinyl)methylamino]propyl]tetrahydro-2-furancarboxamide, having the following structural formula II(a):

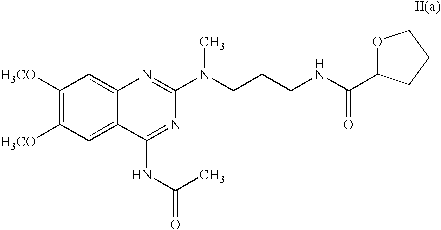

37. N-Propionyl substituted compound of alfuzosin, N-[3-[(4-propionylamino-6,7-dimethoxy-2-quinazolinyl)methylamino]propyl]tetrahydro-2-furancarboxamide, having the following structural formula II(b):

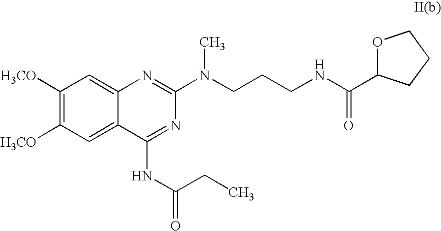

38. A process for preparing highly pure alfuzosin hydrochloride without isolating alfuzosin base as a solid, comprising:
a) reacting N-(4-amino-6,7-dimethoxyquinazol-2-yl)-N-methylpropylenediamine with tetrahydro-2-furoic acid in the presence of N,N-carbonyldiimidazole in a suitable organic solvent to obtain a solution containing alfuzosin free base;
b) optionally, filtering the solution obtained in step-(a) to remove any extraneous matter;
c) optionally, partially or completely concentrating the solution obtained in step-(a) or step-(b) to produce a solution containing alfuzosin free base and the suitable solvent;
d) reacting the alfuzosin solution obtained in step-(a), step-(b) or step-(c) with an acylating agent selected from acetic anhydride or propionic anhydride to produce appropriate N-acyl alfuzosin;
e) hydrolyzing the N-acyl alfuzosin obtained in step-(d) with methanolic hydrochloride in an alcoholic solvent to produce a reaction mass containing alfuzosin hydrochloride; and
f) isolating pure alfuzosin hydrochloride.

39. The process of claim 38, wherein the organic solvent used in step-(a) is selected from the group consisting of hydrocarbons, chlorinated hydrocarbons, and mixtures thereof, wherein the reaction in step-(d) is carried out in the presence a solvent selected from the group consisting of hydrocarbons, chlorinated hydrocarbons, and mixtures thereof, and wherein the solvent used in step-(e) is selected from the group consisting of water, alcohols ketones, cyclic ethers, aliphatic ethers chlorinated hydrocarbons, nitriles, esters and the like, and mixtures thereof.

40. The process of claim 39, wherein the organic solvent used in step-(a) is methylene chloride, wherein the solvent used in step-(d) is selected from the group consisting of hexane, heptane, cyclohexane, toluene, methylene chloride, and mixtures thereof, and wherein the solvent used in step-(e) is selected from the group consisting of water, methanol, ethanol, isopropyl tert-butanol, acetone and mixtures thereof.

41. (canceled)

42. (canceled)

43. (canceled)

44. The process of claim 38, wherein the acylating agent in step-(d) is used in a molar ratio of about 1 to 6 moles per 1 mole of alfuzosin.

45. (canceled)

46. The process of claim 38, wherein the hydrolysis in step-(e) is carried out at a temperature of about 25° C. to the reflux temperature of the solvent used, and wherein the isolation in step-(f) is initiated by cooling, seeding, partial removal of the solvent from the solution, by adding an anti-solvent to the solution or a combination thereof.

47. (canceled)

48. (canceled)

49. (canceled)

50. The process of claim 46, wherein the isolation is carried out by cooling the solution at a temperature below 30° C.

51. (canceled)

52. (canceled)
